(19) United States
(12) Patent Application Publication
(10) Pub. No.: US 2025/0261615 A1
(43) Pub. Date: Aug. 21, 2025

(54) ABSORBENT ARTICLE FOR PET

(71) Applicant: Unicharm Corporation, Shikokuchuo-shi (JP)

(72) Inventor: Daisuke KOMATSUBARA, Kanonji-shi (JP)

(21) Appl. No.: 19/201,375

(22) Filed: May 7, 2025

Related U.S. Application Data
(63) Continuation of application No. PCT/JP2023/035220, filed on Sep. 27, 2023.

(30) Foreign Application Priority Data
Nov. 14, 2022 (JP) 2022-181966

(51) Int. Cl. A01K 23/00

(52) U.S. Cl. CPC A01K 23/00

(57) ABSTRACT

An absorbent article for a pet according to one aspect is an absorbent article for a pet that is wound around a body of a pet to be worn. The absorbent article includes a body portion having a laterally long shape having a longitudinal direction and a lateral direction. The body portion includes a front surface sheet disposed on a skin surface side, a back surface sheet disposed on a non-skin surface side, an absorber disposed between the front surface sheet and the back surface sheet, and a pair of leakage-preventing gathers disposed outside the absorber in the lateral direction. At least a part of one or both of the pair of leakage-preventing gathers is colored in a color different from the front surface sheet.

### TECHNICAL FIELD

The present disclosure relates to an absorbent article for a pet.

### BACKGROUND ART

Patent Literature 1 discloses a belly-wound absorbent article for a pet wound around a body of a pet such as a dog or a cat to be worn. The belly-wound absorbent article for a pet is suitably used for a male pet in which a urethral opening is positioned in front of a root of a hind leg, and has a laterally long shape including a liquid-permeable front surface sheet, a liquid-impermeable back surface sheet, an absorber disposed between the front surface sheet and the back surface sheet, and a pair of leakage-preventing gathers disposed outside the absorber. Urine of the pet is absorbed by winding the absorbent article for a pet around the body of the pet to be worn so as to cover the urethral opening of the pet.

### CITATION LIST

### Patent Literature

Patent Literature 1: Japanese Patent No. 5624925

### SUMMARY OF INVENTION

### Technical Problem

In the absorbent article for a pet described above, it is required to put the absorbent article for a pet such that the urethral opening of the pet is disposed between the pair of leakage-preventing gathers. For example, in a case where the absorbent article for a pet is worn on the male pet, it is required that one leakage-preventing gather of the pair of leakage-preventing gathers is disposed in front of the pet (head side) with respect to the urethral opening and the other leakage-preventing gather of the pair of leakage-preventing gathers is disposed behind the pet (hip side) with respect to the urethral opening, and more preferably, is disposed between a root of a genital organ of the pet and testicles. In a case where the urethral opening of the pet is not disposed between the pair of leakage-preventing gathers, the urine of the pet is not absorbed by the absorber, and urine leakage occurs.

However, a user may not recognize a correct method of putting the absorbent article for a pet. Even in a case where the correct putting method is recognized, when the absorbent article for a pet is worn on the pet, it is necessary to put the absorbent article for a pet while looking into a position of the urethral opening from a side of the pet, and it is not easy to put the absorbent article for a pet at an appropriate position.

Accordingly, an object of the present disclosure is to provide an absorbent article for a pet that is easily worn at an appropriate position.

### Solution to Problem

In one aspect, there is provided an absorbent article for a pet wound around a body of a pet to be worn. The absorbent article for a pet is an absorbent article for a pet wound around a body of a pet to be worn, and the absorbent article includes a body portion having a laterally long shape having a longitudinal direction and a lateral direction. The body portion includes a front surface sheet disposed on a skin surface side, a back surface sheet disposed on a non-skin surface side, an absorber disposed between the front surface sheet and the back surface sheet, and a pair of leakage-preventing gathers disposed outside the absorber in the lateral direction, and at least a part of one or both of the pair of leakage-preventing gathers is colored in a color different from the front surface sheet.

In the above aspect, since at least a part of the leakage-preventing gather is colored in a color different from the front surface sheet, the user can easily recognize the position of the leakage-preventing gather. As a result, it becomes easy to put the absorbent article for a pet while confirming a positional relationship between the urethral opening of the pet and the leakage-preventing gather, and as a result, the urethral opening of the pet can be disposed between the pair of leakage-preventing gathers. Accordingly, it is easy to attach the absorbent article for a pet at an appropriate position.

### Advantageous Effects of Invention

According to various aspects of the present invention, the absorbent article for a pet is easily worn at an appropriate position.

### BRIEF DESCRIPTION OF DRAWINGS

FIG. 1 is a perspective view schematically illustrating an absorbent article for a pet according to one embodiment.

FIG. 2 is a plan view of the absorbent article for a pet as viewed from a skin surface side.

FIG. 3 is a plan view of the absorbent article for a pet as viewed from a non-skin surface side.

FIG. 4 is a sectional view of the absorbent article for a pet taken along line A-A in FIG. 2.

FIG. 5 is a sectional view of the absorbent article for a pet taken along line B-B in FIG. 1.

FIG. 6 is a sectional view of the absorbent article for a pet taken along line C-C in FIG. 2.

FIG. 7 is a sectional view of the absorbent article for a pet taken along line D-D in FIG. 2.

FIG. 8 is a diagram for explaining a procedure for putting the absorbent article for a pet on a pet.

FIG. 9 is a diagram illustrating a position where a rear leakage-preventing gather is to be disposed.

FIG. 10 is a diagram illustrating a procedure subsequent to FIG. 8.

FIG. 11 is a diagram illustrating a procedure subsequent to FIG. 10.

FIG. 12 is a plan view of an absorbent article for a pet according to another embodiment as viewed from a skin surface side.

FIG. 13 is a plan view of an absorbent article for a pet according to still another embodiment as viewed from a skin surface side.

### DESCRIPTION OF EMBODIMENTS

### Overview of Embodiments of Present Disclosure

An overview of embodiments of the present disclosure will be described with reference to the following clauses. Note that, the present disclosure includes any combination of the following clauses.

### Clause 1

An absorbent article for a pet that is wound around a body of a pet to be worn, the absorbent article including a body portion having a laterally long shape having a longitudinal direction and a lateral direction, in which the body portion includes a front surface sheet disposed on a skin surface side, a back surface sheet disposed on a non-skin surface side, an absorber disposed between the front surface sheet and the back surface sheet, and a pair of leakage-preventing gathers disposed outside the absorber in the lateral direction, and at least a part of one or both of the pair of leakage-preventing gathers is colored in a color different from the front surface sheet.

In the above aspect, since at least apart of the leakage-preventing gather is colored in a color different from the front surface sheet, the user can easily recognize the position of the leakage-preventing gather. As a result, it becomes easy to put the absorbent article for a pet while confirming a positional relationship between the urethral opening of the pet and the leakage-preventing gather, and as a result, the urethral opening of the pet can be disposed between the pair of leakage-preventing gathers. Accordingly, it is easy to attach the absorbent article for a pet at an appropriate position.

### Clause 2

In the absorbent article for a pet according to Clause 1, the body portion may have a front edge disposed on one side in the lateral direction and a rear edge disposed on the other side in the lateral direction, the pair of leakage-preventing gathers may include a front leakage-preventing gather provided along the front edge and a rear leakage-preventing gather provided along the rear edge, and at least a part of the rear leakage-preventing gather may be colored in a color different from the front surface sheet. In the present aspect, since at least a part of the rear leakage-preventing gather is colored in a color different from the front surface sheet, the user can easily recognize the position of the rear leakage-preventing gather when the absorbent article for a pet is worn. Accordingly, the rear leakage-preventing gather is easily disposed at an appropriate position.

### Clause 3

In the absorbent article for a pet according to Clause 2, each of the front leakage-preventing gather and the rear leakage-preventing gather may include a side sheet partially fixed to the front surface sheet or the back surface sheet, and a first elastic member disposed along an inner edge of the side sheet in the lateral direction and joined to the side sheet in a state of being stretched in the longitudinal direction, and at least a partial region of the side sheet of the rear leakage-preventing gather may be colored in a color different from the front surface sheet. Since at least a part of the region of the side sheet is colored in a color different from the front surface sheet, the position of the rear leakage-preventing gather can be easily recognized by the user when the absorbent article for a pet is worn.

### Clause 4

In the absorbent article for a pet according to Clause 3, a length of the colored region in the longitudinal direction may be ¼ or more of an entire length of the body portion in the longitudinal direction. The length of the colored region in the longitudinal direction is set to ¼ or more of the entire length of the body portion in the longitudinal direction, the length of the colored region becomes longer than a body width of the pet. Accordingly, when the absorbent article for a pet is worn, the colored region can be visually recognized from above the pet. Accordingly, the position of the rear leakage-preventing gather can be recognized by the user.

### Clause 5

In the absorbent article for a pet according to Clause 3 or 4, the colored region may be formed so as to straddle the absorber in the longitudinal direction. According to the present aspect, the position of the rear leakage-preventing gather can be recognized by the user.

### Clause 6

In the absorbent article for a pet according to any one of Clauses 3 to 5, an inner portion of the side sheet in the lateral direction may be folded back, and the first elastic member may be disposed between the folded side sheets. The inner portion of the side sheet is folded back, and thus, the side sheet partially overlaps and the color of the inner edge of the side sheet becomes dark. Accordingly, the position of the inner edge of the rear leakage-preventing gather can be more reliably recognized by the user.

### Clause 7

In the absorbent article for a pet according to any one of Clauses 3 to 6, the back surface sheet may include a liquid-impermeable back surface film and a back surface nonwoven fabric disposed on the non-skin surface side with respect to the back surface film, and a width of the back surface film may be narrower than a width of the back surface nonwoven fabric in the lateral direction, and the colored region may extend outward in the lateral direction from the back surface film so as to be visibly recognizable from the non-skin surface side through the back surface nonwoven fabric. In the present aspect, when the absorbent article for a pet is worn, when the rear edge of the body portion is rolled up and caught inside, since the colored region of the side sheet is hidden by the back surface film and cannot be visually recognized, it is possible to allow the user to recognize that the rear edge of the body portion is caught inside. Accordingly, according to the present aspect, urine leakage due to the catching of the rear edge can be suppressed.

### Clause 8

In the absorbent article for a pet according to any one of Clauses 2 to 7, each of the front leakage-preventing gather and the rear leakage-preventing gather may include a side sheet partially fixed to the front surface sheet or the back surface sheet, and a first elastic member disposed along an inner edge of the side sheet in the lateral direction and joined to the side sheet in a state of being stretched in the longitudinal direction, and the first elastic member of the rear leakage-preventing gather may be colored in a color different from the front surface sheet so as to be visibly recognizable through the side sheet. Since the first elastic member is colored so as to be visibly recognizable through the side sheet, the position of the inner edge of the rear leakage-preventing gather can be recognized by the user when the absorbent article for a pet is worn.

### Clause 9

In the absorbent article for a pet according to any one of Clauses 3 to 8, an effective length of the first elastic member may be ¼ or more of an entire length of the body portion in the longitudinal direction. The effective length of the first elastic member is set to ¼ or more of the entire length in the longitudinal direction of the body portion, and thus, the effective length of the first elastic member becomes longer than the body width of the pet. Accordingly, when the absorbent article for a pet is worn, since the colored first elastic member can be visually recognized from above the pet, the position of the rear leakage-preventing gather can be recognized by the user.

### Clause 10

In the absorbent article for a pet according to any one of Clauses 3 to 9, an inner edge of the side sheet of the rear leakage-preventing gather in the lateral direction may not be joined to the front surface sheet over the entire length in the longitudinal direction. The inner edge of the side sheet of the rear leakage-preventing gather is not joined to the front surface sheet over the entire length in the longitudinal direction, and thus, the rear leakage-preventing gather easily falls outward in the lateral direction. The rear leakage-preventing gather falls outward, and thus, the colored first elastic member can be easily visually recognized.

### Clause 11

In the absorbent article for a pet according to any one of Clauses 3 to 9, an inner edge of the side sheet of the rear leakage-preventing gather in the lateral direction may be joined to the front surface sheet in a state of being folded back outward in the lateral direction at both end portions in the longitudinal direction. The inner edge of the side sheet of the rear leakage-preventing gather is joined to the front surface sheet in a state of being folded back to the outside in the lateral direction at both end portions in the longitudinal direction, and thus, the rear leakage-preventing gather easily falls to the outside in the lateral direction. The rear leakage-preventing gather falls outward, and thus, the colored first elastic member can be easily visually recognized.

### Clause 12

In the absorbent article for a pet according to any one of Clauses 3 to 11, the side sheet may include a fixed portion joined to the front surface sheet or the back surface sheet, and a rising portion disposed inside the fixed portion in the lateral direction and capable of rising with an inner edge of the fixed portion as a starting point, and a height of the raised rising portion may be larger than a width between the inner edge of the fixed portion and the rear edge in the lateral direction. The height of the rising portion is set to be larger than the width between the inner edge and the rear edge of the fixed portion, and thus, when the absorbent article for a pet is worn in a state where the rising portion rises, the colored region extends outward from the rear edge and becomes visibly recognizable from the outside. The user can confirm that the absorbent article for a pet is appropriately worn by visually recognizing the rising portion. Accordingly, the user has confidence in appropriate putting the absorbent article for a pet by visually recognizing the rising portion, and can eliminate the anxiety about the urine leakage.

### Clause 13

In the absorbent article for a pet according to Clause 12, the back surface sheet may include a liquid-impermeable back surface film and a back surface nonwoven fabric disposed on the non-skin surface side with respect to the back surface film, and a width of the back surface film may be narrower than a width of the back surface nonwoven fabric in the lateral direction, and a height of the raised rising portion may be larger than a width between the inner edge of the fixed portion and an outer edge of the back surface film in the lateral direction. The height of the rising portion is set to be larger than the width between the fixed portion and the outer edge of the back surface film in the lateral direction, and thus, the rising portion can be visually recognized from the outside when the absorbent article for a pet is worn. Accordingly, the absorbent article for a pet can be attached at an appropriate position.

### Clause 14

The absorbent article for a pet according to any one of Clauses 3 to 13 may further include a pair of second elastic members disposed outside the first elastic member in the lateral direction and extending along the front edge and the rear edge of the body portion, respectively, in which the second elastic member extending along the rear edge, of the pair of second elastic members, may be colored in a color different from the front surface sheet. In the present aspect, the colored second elastic member becomes visibly recognizable when the absorbent article for a pet is appropriately worn. On the other hand, when the rear edge of the body portion is caught inside, since the colored second elastic member is hidden and cannot be visually recognized, the user can recognize that the rear edge of the body portion is caught inside.

### Clause 15

In the absorbent article for a pet according to Clause 14, the back surface sheet may include a liquid-impermeable back surface film and a back surface nonwoven fabric disposed on the non-skin surface side with respect to the back surface film, and a width of the back surface film may be narrower than a width of the back surface nonwoven fabric in the lateral direction, and the pair of second elastic members may be joined to the back surface nonwoven fabric outside he back surface film in the lateral direction so as to be visibly recognizable from the non-skin surface side through the back surface nonwoven fabric. In the present aspect, the colored second elastic member can be visually recognized through the back surface nonwoven fabric.

### Clause 16

In the absorbent article for a pet according to any one of Clauses 2 to 15, at least a part of the rear leakage-preventing gather may be housed in a package in a folded state so as to be visibly recognizable from an outside. In the present aspect, the user can recognize the front and back of the absorbent article for a pet before the absorbent article for a pet is spread.

### DETAILS OF EMBODIMENTS OF PRESENT DISCLOSURE

Hereinafter, the embodiments of the present disclosure will be described in detail with reference to the drawings. In the description of the drawings, identical elements are denoted by identical reference signs, and redundant description will be omitted. The drawings may be partially simplified or exaggerated for ease of understanding, and dimensional ratios are not limited to those described in the drawings.

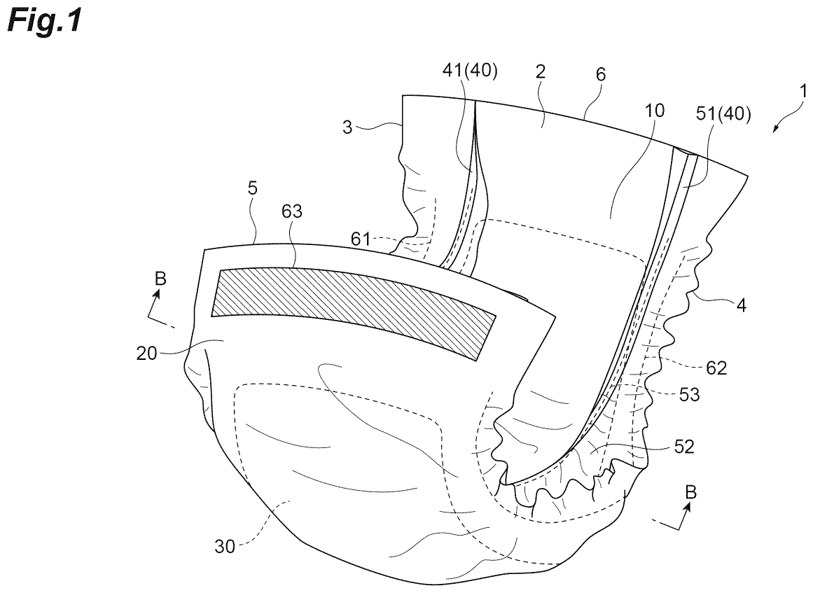

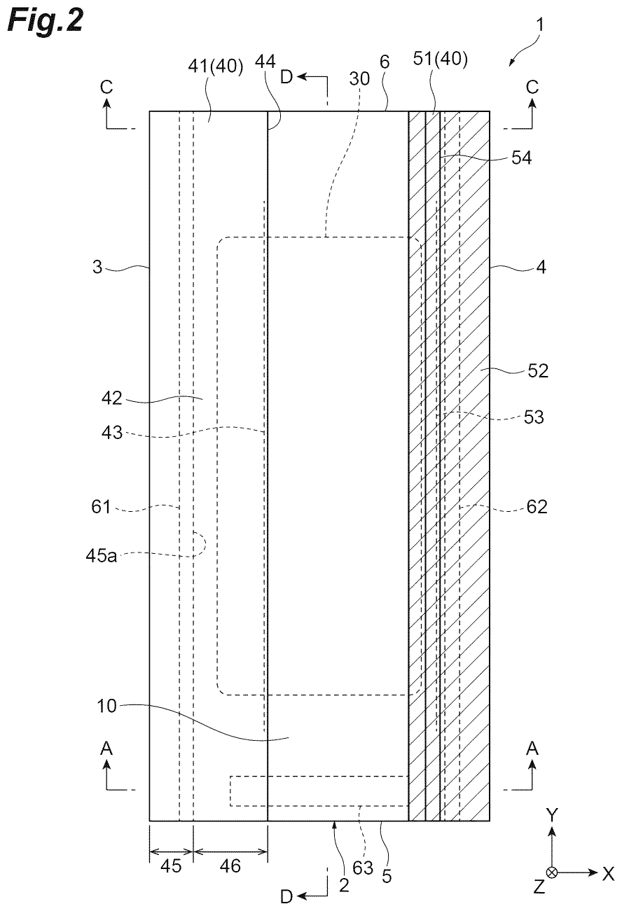

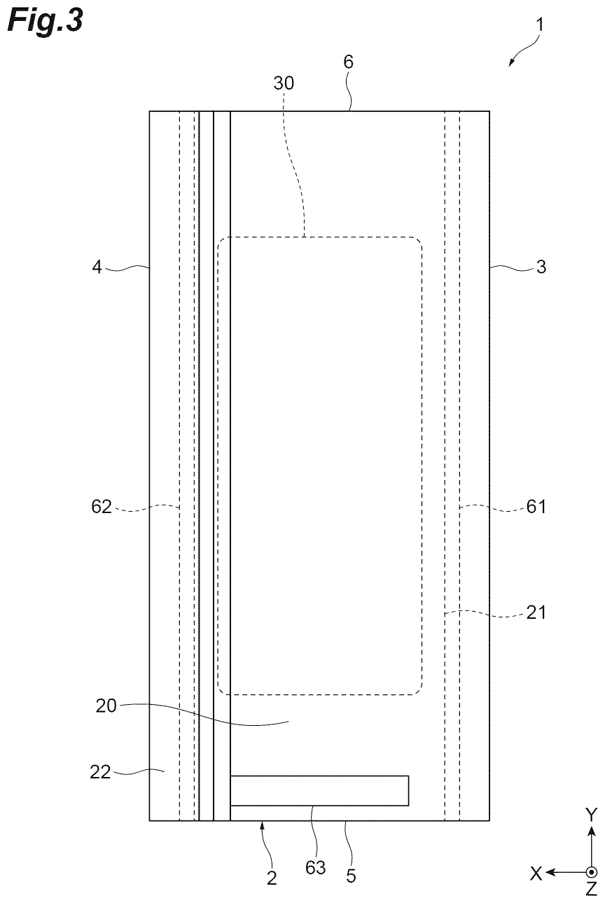

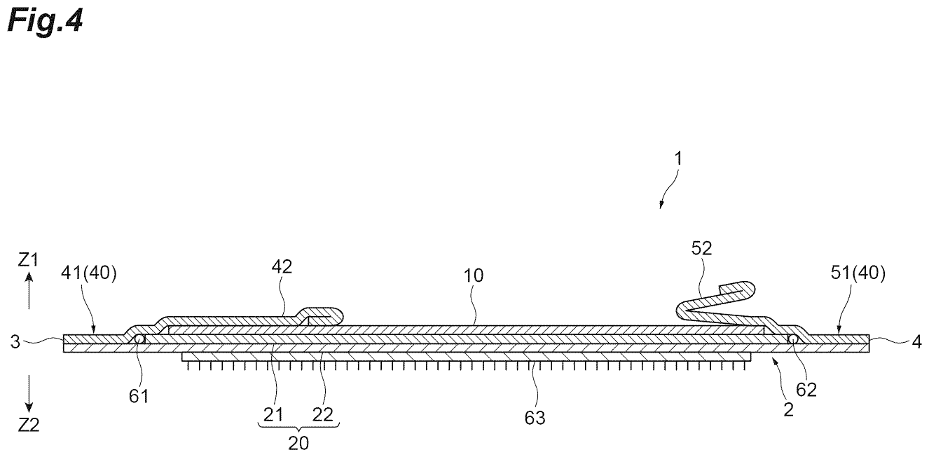

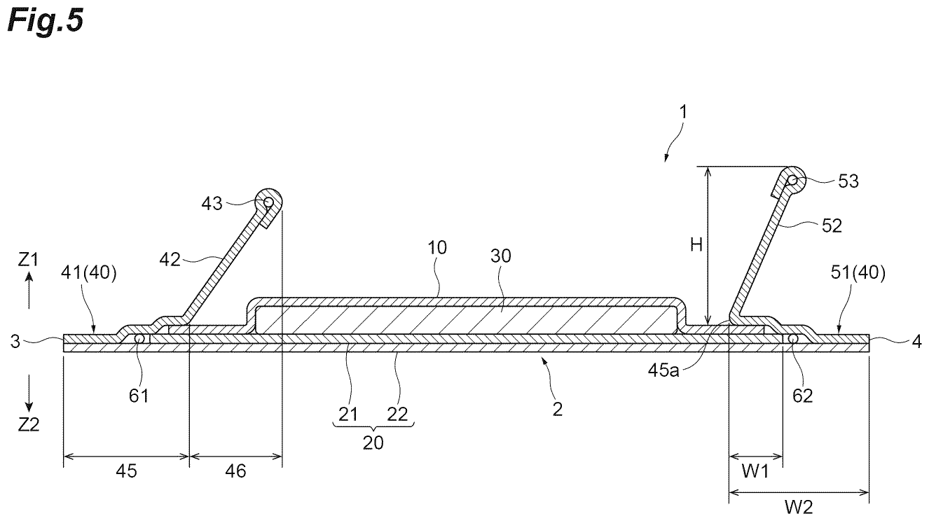

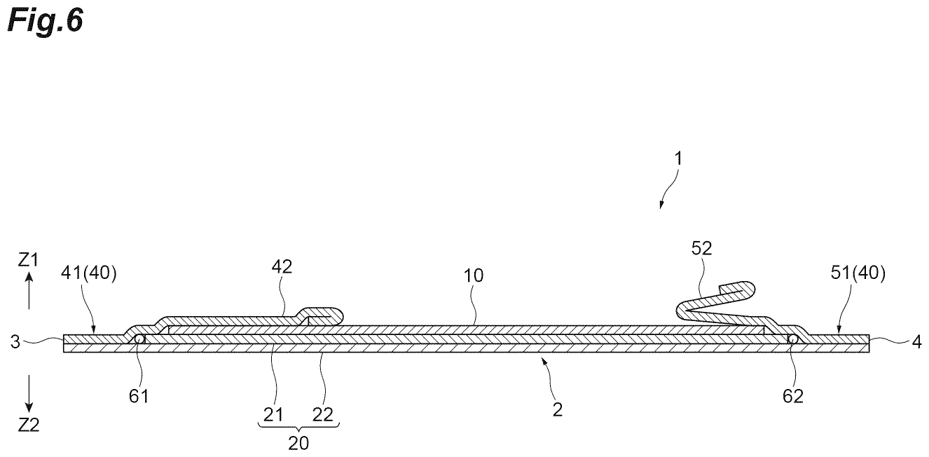

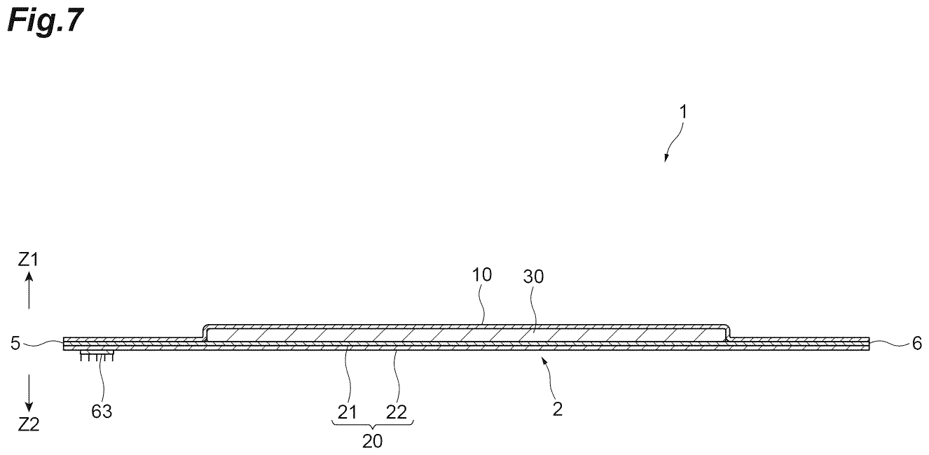

Hereinafter, an absorbent article for a pet according to one embodiment will be described. FIG. 1 is a perspective view of an absorbent article for a pet 1 according to one embodiment. FIG. 2 is a plan view of the absorbent article for a pet 1 as viewed from a skin surface side Z1, and FIG. 3 is a plan view of the absorbent article for a pet 1 as viewed from a non-skin surface side Z2. FIG. 4 is a sectional view of the absorbent article for a pet 1 taken along line A-A in FIG. 2. FIG. 5 is a sectional view of the absorbent article for a pet 1 taken along line B-B in FIG. 1. FIG. 6 is a sectional view of the absorbent article for a pet 1 taken along line C-C in FIG. 2. FIG. 7 is a sectional view of the absorbent article for a pet 1 taken along line D-D in FIG. 2. The absorbent article for a pet 1 illustrated in FIGS. 1 to 7 is a pet disposable diaper that absorbs urine of a pet.

FIGS. 2 and 3 illustrate the absorbent article for a pet 1 in an unfolded state where the absorbent article for a pet 1 is stretched to a state where wrinkles are not formed. The skin surface side Z1 is a side directed to a skin surface of the pet when the absorbent article for a pet 1 is in a putting state, and the non-skin surface side Z2 is a side directed to a side opposite to the skin surface of the pet when the absorbent article for a pet 1 is in the putting state. In the following description, a person who puts the absorbent article for a pet 1 is referred to as a “user”. Typically, the user is an owner of the pet. In the following description, a positional relationship in the unfolded state will be described unless otherwise specified.

In the present specification, pets broadly include vertebrates and invertebrates, and typically include pets such as dogs, cats, rabbits, and hamsters. The absorbent article for a pet 1 is particularly suitably used for male pets in which a urethral opening is positioned in front of a root of a hind leg. As illustrated in FIGS. 2 and 3, the absorbent article for a pet 1 extends in a lateral direction X disposed along a front-back direction (direction connecting the head and the hips) of the pet and in a longitudinal direction Y disposed along a body direction of the pet. The lateral direction X and the longitudinal direction Y are directions perpendicular to each other. A thickness direction Z is a direction perpendicular to the lateral direction X and the longitudinal direction Y, and includes the skin surface side Z1 directed to the skin surface of the pet in the putting state and the non-skin surface side Z2 directed to the side opposite to the skin surface of the pet in the putting state. The absorbent article for a pet 1 is worn by being wound around the body of the pet.

The absorbent article for a pet 1 includes a body portion 2. The body portion 2 has a substantially rectangular (laterally long) planar shape having the lateral direction X and the longitudinal direction Y. The body portion 2 has a front edge 3 disposed on one end side in the lateral direction X and a rear edge 4 disposed on the other end side in the lateral direction X. When the absorbent article for a pet 1 is worn on the pet, the front edge 3 is disposed on a front leg side of the pet, and the rear edge 4 is disposed on a hind leg side of the pet. In the following description, a direction close to the front edge 3 in the lateral direction may be referred to as a front side, and a direction close to the rear edge 4 in the lateral direction may be referred to as a rear side. In addition, the body portion 2 has a first side edge 5 disposed on one end side in the longitudinal direction Y and a second side edge 6 disposed on the other end side in the longitudinal direction Y.

As illustrated in FIGS. 1 to 6, the body portion 2 includes a front surface sheet 10, a back surface sheet 20, an absorber 30, and a pair of leakage-preventing gathers 40. The front surface sheet 10 constitutes a surface of the body portion 2 that is brought into contact with the pet, and is disposed on the skin surface side Z1. The front surface sheet 10 is liquid permeability that allows the urine of the pet to permeate to the absorber 30 side, for example. The front surface sheet 10 is positioned substantially at the center in the lateral direction X. Typically, the front surface sheet 10 is a white and plain nonwoven fabric.

The back surface sheet 20 constitutes a surface of the body portion 2 positioned on an outer side when the pet wears the absorbent article for a pet, and is disposed on the non-skin surface side Z2. The back surface sheet 20 includes a liquid-impermeable back surface film 21 and a back surface nonwoven fabric 22 disposed on the non-skin surface side Z2 with respect to the back surface film 21. In the lateral direction X, a width of the back surface film 21 is narrower than a width of the back surface nonwoven fabric 22, and the back surface nonwoven fabric 22 extends to both sides in the lateral direction X with respect to the back surface film 21. The back surface nonwoven fabric 22 constitutes an outermost layer of the absorbent article for a pet 1.

The absorber 30 is disposed between the front surface sheet 10 and the back surface sheet 20. The absorber 30 absorbs the urine of the pet. The absorber 30 has a substantially rectangular shape extending in the longitudinal direction Y. In one embodiment, the absorber 30 includes an absorbent core including pulp having water absorbency and a super-absorbent polymer (SAP), and a core wrap covering a front surface of the absorbent core. For example, chemical pulp, cellulose fiber, and cellulose fiber such as rayon and acetate are used as the pulp of the absorbent core. For example, starch-based, acrylic acid-based, and amino acid-based particulate or fibrous polymers are used as the super-absorbent polymer. Typically, the core wrap is made of white and plain tissue or nonwoven fabric. Note that, the core wrap may be colored.

The pair of leakage-preventing gathers 40 is disposed outside the absorber 30 in the lateral direction X, and suppresses the leakage of the urine of the pet excreted in the absorber 30 in the lateral direction X. The pair of leakage-preventing gathers 40 is disposed so as to cover both side portions in the lateral direction X of the front surface sheet 10 from the skin surface side Z1, and extends in the longitudinal direction Y over an entire length of the body portion 2 in the longitudinal direction Y. The pair of leakage-preventing gathers 40 includes a front leakage-preventing gather 41 provided along the front edge 3 and a rear leakage-preventing gather 51 provided along the rear edge 4. In the following description, in a case where it is not particularly necessary to distinguish these gathers, the front leakage-preventing gather 41 and the rear leakage-preventing gather 51 may be collectively referred to as the leakage-preventing gather 40.

In one embodiment, the front leakage-preventing gather 41 includes a side sheet 42 partially fixed to the front surface sheet 10 or the back surface sheet 20, and a first elastic member 43 joined to the side sheet 42. The rear leakage-preventing gather 51 includes a side sheet 52 partially fixed to the front surface sheet 10 or the back surface sheet 20, and a first elastic member 53 joined to the side sheet 52. Each of the pair of side sheets 42 and 52 is made of a nonwoven fabric having water repellency or hydrophobicity. For example, the side sheets 42 and 52 are made of a spunlace nonwoven fabric, a spunbond nonwoven fabric, a thermal bond nonwoven fabric, a melt-blown nonwoven fabric, an SMS nonwoven fabric, a needle punch nonwoven fabric, an air-through nonwoven fabric, or the like.

Each of the pair of side sheets 42 and 52 has a vertically long shape that is long in the longitudinal direction Y, and extends in the longitudinal direction Y over the entire length of the body portion 2 in the longitudinal direction Y. As illustrated in FIGS. 4 to 6, the pair of leakage-preventing gathers 40 extends outward with respect to the back surface film 21. More specifically, an outer edge of the front leakage-preventing gather 41 in the lateral direction X coincides with one outer edge of the back surface nonwoven fabric 22 in the lateral direction X, and constitutes the front edge 3 together with one outer edge of the back surface nonwoven fabric 22. The outer edge of the rear leakage-preventing gather 51 in the lateral direction X coincides with the other outer edge of the back surface nonwoven fabric 22 in the lateral direction, and constitutes the rear edge 4 together with the other outer edge of the back surface nonwoven fabric 22.

As illustrated in FIG. 5, each of the side sheets 42 and 52 include a fixed portion 45 joined to the front surface sheet 10 or the back surface sheet 20, and a rising portion 46 not joined to the front surface sheet 10. The fixed portion 45 is joined to the front surface sheet 10 or the back surface sheet 20 with, for example, a hot melt adhesive. The rising portion 46 is disposed inside the body portion 2 with respect to the fixed portion 45 in the lateral direction X, and can rise with an inner edge 45a of the fixed portion 45 as a starting point.

As illustrated in FIG. 5, a height H of the rising portion 46 rising with the inner edge 45a of the fixed portion 45 as the starting point is larger than a width W1 between the inner edge 45a in the lateral direction X and the outer edge of the back surface film 21. In addition, the height H of the rising portion 46 is larger than a width W2 between the inner edge 45a of the fixed portion 45 and the rear edge 4 in the lateral direction X. Note that, the height H of the rising portion 46 is a maximum height of the rising portion 46 in the thickness direction Z with the inner edge 45a as a reference.

As illustrated in FIGS. 4 and 6, both end portions of an inner edge 44 of the side sheet 42 of the front leakage-preventing gather 41 in the longitudinal direction Y are joined to the front surface sheet 10. More specifically, the side sheet 42 of the front leakage-preventing gather 41 is joined to the front surface sheet 10 in a state of falling inward in the lateral direction X in the vicinity of the first side edge 5 and the second side edge 6. Portions excluding both the end portions of the inner edge 44 of the side sheet 42 of the front leakage-preventing gather 41 in the longitudinal direction Y are not joined to the front surface sheet 10. Both the end portions of the inner edge 44 of the side sheet 42 of the front leakage-preventing gather 41 in the longitudinal direction Y serve as rising fulcrums of the side sheet 42 in the longitudinal direction Y. As described above, both the end portions of the inner edge 44 of the side sheet 42 of the front leakage-preventing gather 41 in the longitudinal direction Y are joined to the front surface sheet 10 in a state of falling inward in the lateral direction X, and thus, the side sheet 42 of the front leakage-preventing gather 41 falls inward in the lateral direction X in the unfolded state.

On the other hand, an inner edge 54 of the side sheet 52 of the rear leakage-preventing gather 51 is not joined to the front surface sheet 10 over the entire length in the longitudinal direction Y. The inner edge 54 of the side sheet 52 of the rear leakage-preventing gather 51 is not joined to the front surface sheet 10 over the entire length in the longitudinal direction Y, and thus, as illustrated in FIGS. 2, 4, and 6, the side sheet 52 of the rear leakage-preventing gather 51 falls outward in the lateral direction X in the unfolded state. The side sheet 52 falls outward, and thus, a distance between the inner edges 44 and 54 of the pair of leakage-preventing gathers 40 in the lateral direction X increases. As a result, a region where the urethral opening of the pet is disposed is widened. Accordingly, the absorbent article for a pet 1 can be easily worn at an appropriate position, and urine leakage can be suppressed. In addition, the rear leakage-preventing gather 51 falls outward in the unfolded state, and thus, the inner edge 54 of the side sheet 52 of the rear leakage-preventing gather 51 is brought close to the rear edge 4. As a result, the absorbent article for a pet 1 can be easily attached to the pet having the urethral opening near the hind leg.

In addition, as described above, in the unfolded state, the front leakage-preventing gather 41 falls inward in the lateral direction X, and the rear leakage-preventing gather 51 falls outward in the lateral direction X. The user can easily recognize a front-back orientation of the absorbent article for a pet 1 based on a difference in the configuration. As a result, an orientation in which the absorbent article for a pet 1 is worn can be easily understood, and a sense of security can be given to the user.

As illustrated in FIG. 5, inner portions of the side sheets 42 and 52 in the lateral direction X are folded back, and the first elastic members 43 and 53 are disposed between the folded side sheets 42 and 52, respectively. Accordingly, as illustrated in FIG. 2, the first elastic member 43 of the front leakage-preventing gather 41 is disposed along the inner edge 44 of the side sheet 42, and the first elastic member 53 of the rear leakage-preventing gather 51 is disposed along the inner edge 54 of the side sheet 52. The first elastic members 43 and 53 are joined to the side sheets 42 and 52, respectively, in a state of being stretched in the longitudinal direction Y. The first elastic members 43 and 53 are made of, for example, a stretchable material such as rubber or spandex that can contract in a length direction.

Effective lengths of the first elastic members 43 and 53 may be ¼ or more or ½ or more of the entire length of the body portion 2 in the longitudinal direction Y. The effective lengths are lengths of portions of the first elastic members 43 and 53 that actually contract in the unfolded state. As illustrated in FIG. 2, both end portions of the first elastic members 43 and 53 are disposed outside the absorber 30 in the longitudinal direction Y. That is, the first elastic members 43 and 53 are formed so as to straddle the absorber 30 in the longitudinal direction. The first elastic members 43 and 53 contract in the longitudinal direction Y, and thus, the rising portions 46 of the side sheets 42 and 52 rise with the inner edge 45a of the fixed portion 45 as the starting point outside the absorber 30 in the lateral direction X. The rising portions 46 of the pair of leakage-preventing gathers 40 rise, and thus, the urine of the pet is prevented from leaking along the front surface of the front surface sheet 10.

At least a part of one or both of the pair of leakage-preventing gathers 40 is colored in a color different from the front surface sheet 10. For example, only one leakage-preventing gather of the pair of leakage-preventing gathers 40 may be colored, and the other leakage-preventing gather of the pair of leakage-preventing gathers 40 may not be colored. In the embodiment illustrated in FIG. 2, the side sheet 52 of the rear leakage-preventing gather 51 of the pair of leakage-preventing gathers 40 is colored in a color different from the front surface sheet 10. That is, in the absorbent article for a pet 1, only the side sheet 52 of the rear leakage-preventing gather 51 of the pair of leakage-preventing gathers 40 is colored, and the side sheet 42 of the front leakage-preventing gather 41 is not colored. Note that, the coloring means that a desired color is imparted to the pair of leakage-preventing gathers 40 in whole or part by using a colorant such as a pigment or a dye. The different colors mean colors having a different brightness, saturation, or hue to the extent that these colors can be visually distinguished. More specifically, in a case where a color difference quantified based on a CIE1976 color space defined in JISZ8729 or the like is 1.5 or more or 3.0 or more, the colors are different.

A colored region of the side sheet 52 is a hatched region in FIG. 2. That is, in the embodiment illustrated in FIG. 2, the entire region of the side sheet 52 of the rear leakage-preventing gather 51 is colored. Since the colored side sheet 52 extends outward in the lateral direction X with respect to the back surface film 21, the colored side sheet can be visually recognized from the non-skin surface side Z2 through the back surface nonwoven fabric 22. Since the colored side sheet 52 is visually recognizable through the back surface nonwoven fabric 22, the side sheet 52 can be visually recognized even in a state where the absorbent article for a pet 1 is folded. As a result, the front-back orientation of the absorbent article for a pet 1 can be grasped before the absorbent article for a pet 1 is spread. Accordingly, the absorbent article for a pet 1 can be smoothly worn on the pet.

When the absorbent article for a pet 1 is worn on the pet, when the rear edge 4 of the body portion 2 is rolled up and caught inside, the rear leakage-preventing gather 51 is covered with the back surface nonwoven fabric 22, and the rising of the rear leakage-preventing gather 51 may be hindered. When the rear leakage-preventing gather 51 does not rise properly, the urine leakage may occur from this portion. As described above, the colored side sheet 52 visually recognizable through the back surface nonwoven fabric 22, and thus, the user can visually recognize from the outside that the rear leakage-preventing gather 51 rises. As a result, it is possible to confirm whether or not the absorbent article for a pet 1 is appropriately worn. Accordingly, the user's anxiety about the urine leakage can be suppressed. Note that, in a case where the core wrap of the absorber 30 is colored, the side sheet 52 may have a color different from the core wrap. In a case where another sheet member such as an intermediate sheet is provided between the front surface sheet 10 and the absorber 30, the side sheet 52 may have a color different from the other sheet member.

When the front surface sheet 10 and the side sheet 52 have the same color, it is difficult to visually distinguish the front surface sheet 10 and the side sheet 52, and it is difficult for the user to recognize a position of the rear leakage-preventing gather 51 when the absorbent article for a pet 1 is worn. On the other hand, the side sheet 52 of the rear leakage-preventing gather 51 is colored in a color different from the front surface sheet 10, and thus, the position of the rear leakage-preventing gather 51 can be easily recognized. In order to make it easy to distinguish the front surface sheet 10 and the side sheet 52, the side sheet 52 may be colored in a conspicuous color such as red, blue, or green.

As described above, since the inner portion of the side sheet 52 in the lateral direction X is folded back, a single region and a double region disposed on the inner edge 54 side with respect to the single region are formed in the side sheet 52. The double region has a darker color than the single region. Accordingly, the user can grasp a position of the inner edge 54 of the rear leakage-preventing gather 51 from a difference in color between the single region and the double region.

The absorbent article for a pet 1 further includes a pair of second elastic members 61 and 62 and a hook tape 63. The pair of second elastic members 61 and 62 is made of, for example, a stretchable material such as rubber or spandex that can contract in the length direction. The second elastic member 61 is disposed on a front side with respect to the first elastic member 43 and extends in the longitudinal direction Y along the front edge 3 of the body portion 2. The second elastic member 62 is disposed on a rear side with respect to the first elastic member 53 and extends in the longitudinal direction Y along the rear edge 4 of the body portion 2. More specifically, as illustrated in FIG. 5, the pair of second elastic members 61 and 62 is disposed between the pair of leakage-preventing gathers 40 and the back surface nonwoven fabric 22 outside the back surface film 21 in the lateral direction X, respectively, and is joined to the body portion 2 in a state of being stretched in the longitudinal direction Y. The pair of second elastic members 61 and 62 may be provided over the entire length of the body portion 2.

As illustrated in FIGS. 1 to 3, the hook tape 63 has a rectangular shape long in the lateral direction X, and is disposed close to the first side edge 5 of the body portion 2. The hook tape 63 is disposed on the non-skin surface side Z2 of the body portion 2. The hook tape 63 is, for example, a mechanical fastener, and can be engaged with the front surface sheet 10 from the skin surface side Z1.

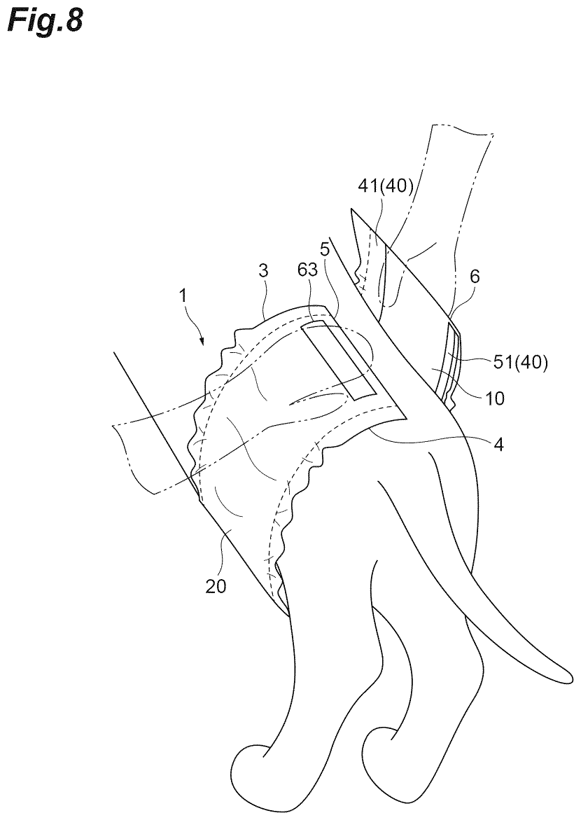

Next, a procedure for put the absorbent article for a pet 1 on the pet will be described with reference to FIGS. 8 to 11. When the absorbent article for a pet 1 is worn on the pet, first, as illustrated in FIG. 8, the user presses the first side edge 5 of the absorbent article for a pet 1 against the back of the pet, and presses the vicinity of the first side edge 5 with one hand. Next, the user holds the vicinity of the second side edge 6 of the absorbent article for a pet 1 with the other hand, and winds the absorbent article for a pet 1 so as to cover the belly of the pet.

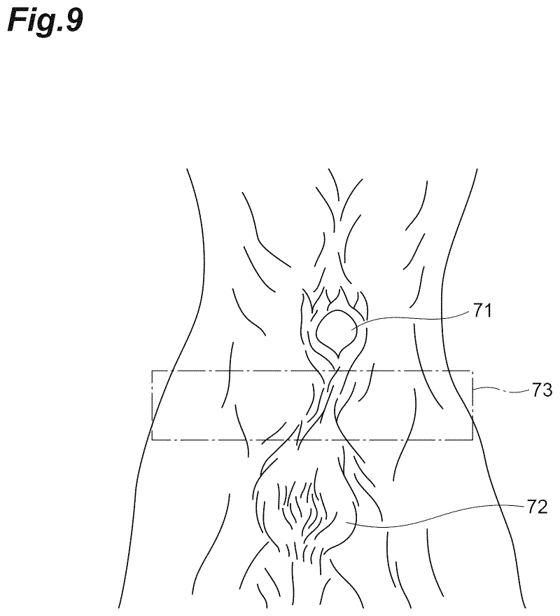

At this time, the absorbent article for a pet 1 is worn such that the front leakage-preventing gather 41 is disposed in front of the pet with respect to the urethral opening and the rear leakage-preventing gather 51 is disposed behind the pet with respect to the urethral opening. For example, in a case where the absorbent article for a pet 1 is worn on a male pet, it is important that a distal end (urethral opening) of the penis is disposed between the pair of leakage-preventing gathers 40. More specifically, as illustrated in FIG. 9, it is preferable that the inner edge 54 of the rear leakage-preventing gather 51 is disposed in a region 73 between a root of a pet penis 71 and testicles 72. The rear leakage-preventing gather 51 is disposed at this position, and thus, the position of the rear leakage-preventing gather 51 is stabilized and hardly deviates. In order to put the absorbent article for a pet 1 at an appropriate position, the user puts the absorbent article for a pet 1 while looking into a position of a genital organ of the pet from the side. At this time, when the front surface sheet 10 of the absorbent article for a pet 1 and the side sheet 52 of the rear leakage-preventing gather 51 have the same color, it is difficult to recognize the position of the inner edge 54 of the rear leakage-preventing gather 51, and it is not easy to put the absorbent article for a pet 1 at an appropriate position. On the other hand, in the absorbent article for a pet 1, since the side sheet 52 of the rear leakage-preventing gather 51 is colored in a color different from the front surface sheet 10, the position of the rear leakage-preventing gather 51 can be easily recognized. Accordingly, it is possible to put the absorbent article for a pet 1 at an appropriate position while confirming a positional relationship between the genital organ of the pet and the rear leakage-preventing gather 51.

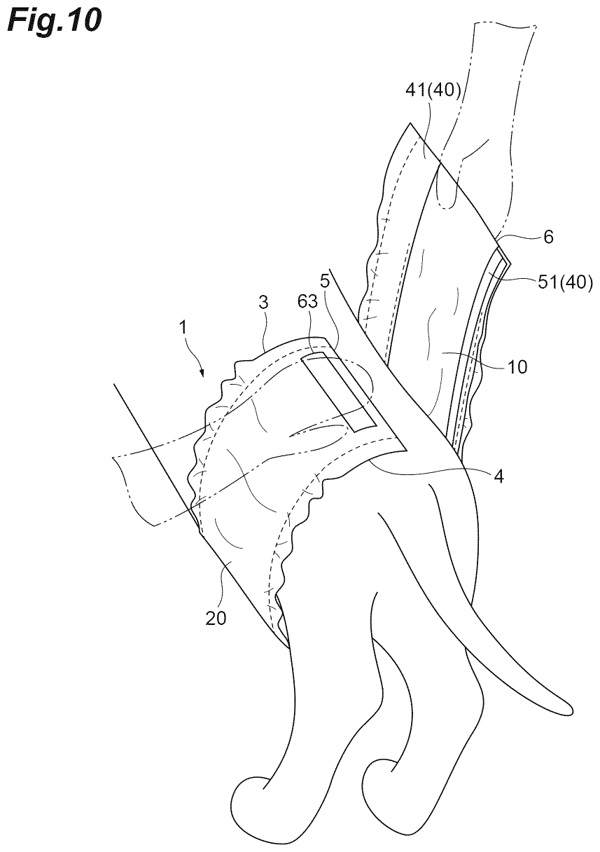

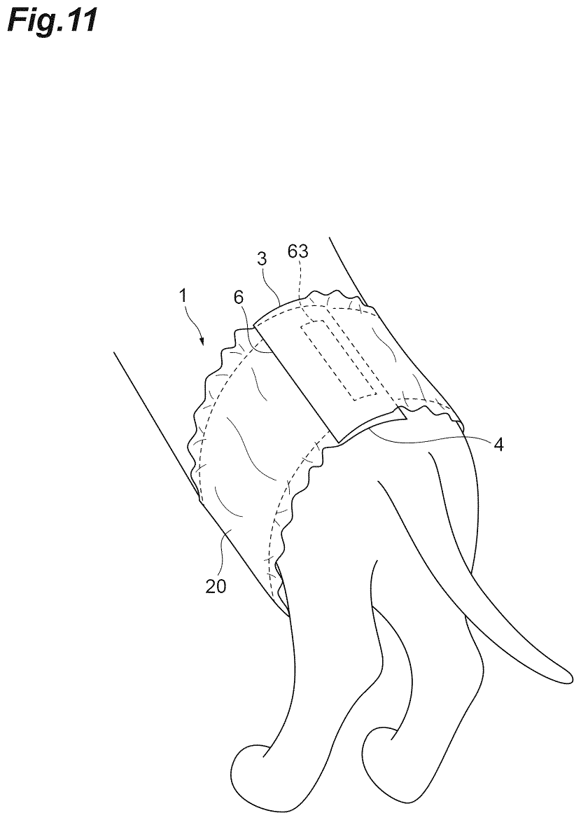

Next, as illustrated in FIG. 10, the user pulls up the second side edge 6 such that the absorbent article for a pet 1 comes into close contact with the body of the pet, and then, as illustrated in FIG. 11, the user engages the front surface sheet 10 with the hook tape 63 in the vicinity of the second side edge 6. As a result, the absorbent article for a pet 1 is worn on the pet at an appropriate position.

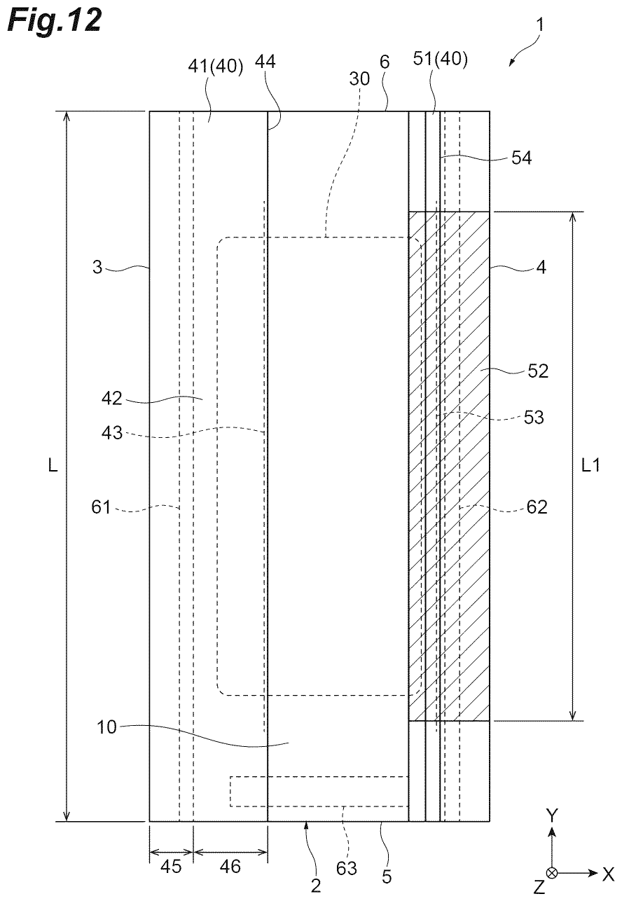

Note that, in one embodiment, as illustrated in FIG. 12, only a part of the side sheet 52 of the rear leakage-preventing gather 51 may be colored in a color different from the front surface sheet 10. In the embodiment illustrated in FIG. 12, the colored region of the side sheet 52 has a length L1 of ¼ or more, or ½ or more of an entire length L of the body portion 2 in the longitudinal direction Y, in the longitudinal direction Y, and is formed so as to straddle the absorber 30. The length of the colored region in the longitudinal direction Y is set to ¼ or more of the entire length L of the body portion 2 in the longitudinal direction Y, and thus, the length L1 of the colored region becomes longer than the body width of the pet. Accordingly, when the user puts the absorbent article for a pet 1, the colored region of the side sheet 52 can be visually recognized from a dorsal side (upper side). As a result, the position of the inner edge 54 of the side sheet 52 can be easily confirmed, and as a result, whether or not the rear leakage-preventing gather 51 is disposed at an appropriate position with respect to the urethral opening can be easily recognized. Note that, an area of the colored region of the side sheet 52 may be ¼ or more, ½ or more, or ¾ or more with respect to an entire area of the side sheet 52.

As described above, in the absorbent article for a pet 1, since at least a part of the side sheet 52 of the rear leakage-preventing gather 51 is colored in a color different from the front surface sheet 10, the user can easily recognize the position of the rear leakage-preventing gather 51. Accordingly, the absorbent article for a pet 1 can be worn while confirming a positional relationship between the urethral opening of the pet and the rear leakage-preventing gather 51 such that the urethral opening of the pet is disposed between the pair of leakage-preventing gathers 40. Thus, the absorbent article for a pet 1 can be worn at an appropriate position.

In a case where the configurations of the pair of leakage-preventing gathers 40 are different as described above, specifically, in a case where the side sheet 42 of the front leakage-preventing gather 41 falls inward in the lateral direction X and the side sheet 52 of the rear leakage-preventing gather 51 falls outward in the lateral direction X in the unfolded state, it is necessary to put the front and back of the absorbent article for a pet 1 in a correct orientation. By contrast, in the absorbent article for a pet 1, since the rear leakage-preventing gather 51 is colored and the front leakage-preventing gather 41 is not colored, the front-back orientation of the absorbent article for a pet 1 can be easily grasped.

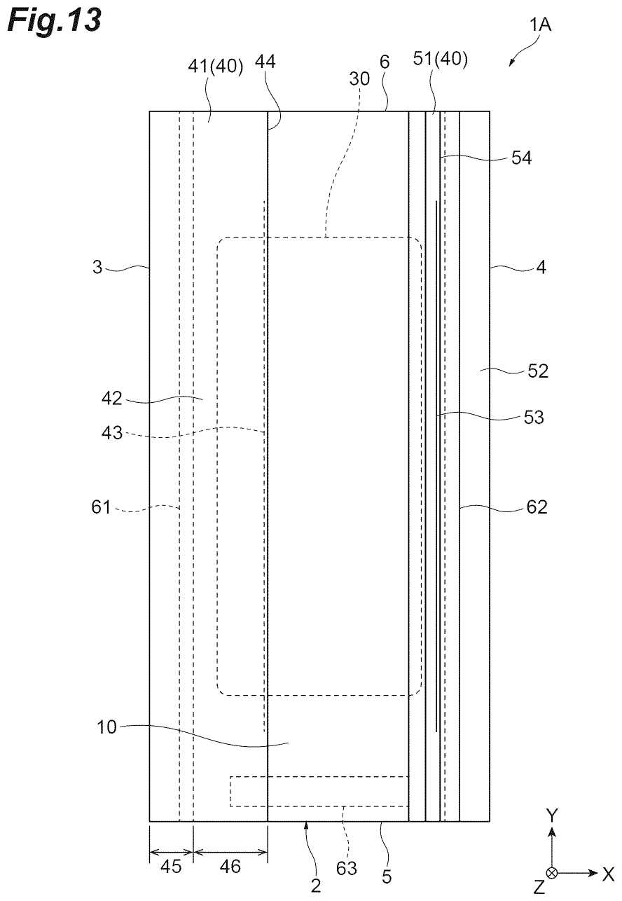

Next, an absorbent article for a pet according to another embodiment will be described. FIG. 13 is a plan view of an absorbent article for a pet 1A according to another embodiment as viewed from the skin surface side Z1. The absorbent article for a pet 1A illustrated in FIG. 13 is different from the absorbent article for a pet 1 illustrated in FIG. 1 in that the first elastic member 53 of the rear leakage-preventing gather 51 is colored in a color different from the front surface sheet 10. In the following description, differences from the above-described absorbent article for a pet 1 will be mainly described, and redundant description will be omitted.

In the absorbent article for a pet 1A, the side sheet 52 of the rear leakage-preventing gather 51 is not colored and has a similar color to the front surface sheet 10. On the other hand, the first elastic member 53 of the rear leakage-preventing gather 51 is colored in a color different from the front surface sheet 10 so as to be visibly recognizable through the side sheet 52. Since the first elastic member 53 of the rear leakage-preventing gather 51 extends along the inner edge 54 of the side sheet 52, when the colored first elastic member 53 can be visually recognized through the side sheet 52, the position of the inner edge 54 of the rear leakage-preventing gather 51 can be recognized. Similarly to the absorbent article for a pet 1 illustrated in FIG. 2, in the absorbent article for a pet 1A, since the positional relationship between the genital organ of the pet and the inner edge 54 of the rear leakage-preventing gather 51 can be easily recognized, the absorbent article for a pet 1 can be worn at an appropriate position.

As described above, since the effective length of the first elastic member 53 is ¼ or more of the entire length of the body portion 2 in the longitudinal direction Y, the length of the colored first elastic member 53 is longer than the body width of the pet. Accordingly, when the absorbent article for a pet 1A is worn, the user can visually recognize the colored first elastic member 53 from the dorsal side (upper side). As a result, it is possible to easily recognize whether or not the rear leakage-preventing gather 51 is disposed at an appropriate position with respect to the urethral opening.

In one embodiment, the second elastic member 62 extending along the rear edge 4 may be colored in a color different from the front surface sheet 10 so as to be visibly recognizable from the skin surface side Z1 or the non-skin surface side Z2 through the back surface nonwoven fabric 22. When the absorbent article for a pet 1A is worn on the pet, when the rear edge 4 of the body portion 2 is rolled up and caught inside, the urine leakage may occur from the caught portion. When the second elastic member 62 is colored in a color different from the side sheet 52, when the rear edge 4 of the body portion 2 is caught inside, the colored second elastic member 62 is hidden behind the back surface film 21 and cannot be seen. As a result, the user can recognize that the rear edge 4 of the body portion 2 is caught inside. Thus, the absorbent article for a pet 1A can be appropriately worn.

Although the absorbent articles for a pet according to various embodiments have been described above, the present invention is not limited to the above-described embodiments, and various modifications can be made without changing the gist of the invention. That is, it should be noted that the above-described embodiments are for the purpose of illustration and are not intended to limit the scope of the present invention.

For example, in the above-described embodiments, although only the side sheet 52 or the first elastic member 53 of the rear leakage-preventing gather 51 is colored, both the side sheet 42 or the first elastic member 43 of the front leakage-preventing gather 41 and the side sheet 52 or the first elastic member 53 of the rear leakage-preventing gather 51 may be colored in a color different from the front surface sheet 10. In addition, in one embodiment, the side sheet 52 or the first elastic member 53 of the rear leakage-preventing gather 51 may not be colored, and the side sheet 42 or the first elastic member 43 of the front leakage-preventing gather 41 may be colored in a color different from the front surface sheet 10. Even in this case, since a position of the front leakage-preventing gather 41 can be easily recognized, the user can easily recognize the positional relationship between the urethral opening of the pet and the front leakage-preventing gather 41 when the absorbent articles for a pet 1 and 1A are worn. Accordingly, the absorbent articles for a pet 1 and 1A can be worn at appropriate positions.

In the embodiment illustrated in FIG. 2, the inner edge 54 of the side sheet 52 of the rear leakage-preventing gather 51 is not joined to the front surface sheet 10 over the entire length in the longitudinal direction Y. However, in one embodiment, the inner edge 54 of the side sheet 52 of the rear leakage-preventing gather 51 in the lateral direction X may be joined to the front surface sheet 10 in a state of being folded back outward in the lateral direction X at both end portions in the longitudinal direction Y. Both end portions of the inner edge 54 of the side sheet 52 of the rear leakage-preventing gather 51 are joined to the front surface sheet 10 in a state of being folded back outward, and thus, the side sheet 52 of the rear leakage-preventing gather 51 easily falls outward in the lateral direction X in the unfolded state.

In the absorbent articles for a pet 1 and 1A, the side sheet 52 or the first elastic member 53 of the rear leakage-preventing gather 51 may be colored, and the colored side sheet 52 or the first elastic member 53 may be housed in a package in a folded state so as to be visibly recognizable from the outside. The colored portion is folded so as to be visibly recognizable from the outside, and thus, the user can recognize the front and back of the absorbent articles for a pet 1 and 1A before the absorbent articles for a pet 1 and 1A are spread. Accordingly, the absorbent articles for a pet 1 and 1A taken out from the package can be smoothly worn on the pet in an appropriate orientation. The various embodiments described above can be combined within a consistent range.

### REFERENCE SIGNS LIST

1, 1A absorbent article for pet
2 body portion
3 front edge
4 rear edge
10 front surface sheet
20 back surface sheet
21 back surface film
22 back surface nonwoven fabric
30 absorber
40 leakage-preventing gather
41 front leakage-preventing gather
42, 52 side sheet
43, 53 first elastic member
44, 54 inner edge
45 fixed portion
46 rising portion
51 rear leakage-preventing gather
61, 62 second elastic member
X lateral direction
Y longitudinal direction
Z1 skin surface side
Z2 non-skin surface side

1. An absorbent article for a pet that is wound around a body of a pet to be worn, the absorbent article comprising:
a body portion having a laterally long shape having a longitudinal direction and a lateral direction,
wherein the body portion includes
a front surface sheet disposed on a skin surface side,
a back surface sheet disposed on a non-skin surface side,
an absorber disposed between the front surface sheet and the back surface sheet, and
a pair of leakage-preventing gathers disposed outside the absorber in the lateral direction, and
at least a part of one or both of the pair of leakage-preventing gathers is colored in a color different from the front surface sheet.

2. The absorbent article for a pet according to claim 1,
wherein the body portion has a front edge disposed on one side in the lateral direction and a rear edge disposed on the other side in the lateral direction,
the pair of leakage-preventing gathers includes a front leakage-preventing gather provided along the front edge and a rear leakage-preventing gather provided along the rear edge, and
at least a part of the rear leakage-preventing gather is colored in a color different from the front surface sheet.

3. The absorbent article for a pet according to claim 2,
wherein each of the front leakage-preventing gather and the rear leakage-preventing gather includes a side sheet partially fixed to the front surface sheet or the back surface sheet, and a first elastic member disposed along an inner edge of the side sheet in the lateral direction and joined to the side sheet in a state of being stretched in the longitudinal direction, and
at least a partial region of the side sheet of the rear leakage-preventing gather is colored in a color different from the front surface sheet.

4. The absorbent article for a pet according to claim 3, wherein a length of the colored region in the longitudinal direction is ¼ or more of an entire length of the body portion in the longitudinal direction.

5. The absorbent article for a pet according to claim 3, wherein the colored region is formed so as to straddle the absorber in the longitudinal direction.

6. The absorbent article for a pet according to claim 3,
wherein an inner portion of the side sheet in the lateral direction is folded back, and
the first elastic member is disposed between the folded side sheets.

7. The absorbent article for a pet according to claim 3,
wherein the back surface sheet includes a liquid-impermeable back surface film and a back surface nonwoven fabric disposed on the non-skin surface side with respect to the back surface film, and a width of the back surface film is narrower than a width of the back surface nonwoven fabric in the lateral direction, and
the colored region extends outward in the lateral direction from the back surface film so as to be visibly recognizable from the non-skin surface side through the back surface nonwoven fabric.

8. The absorbent article for a pet according to claim 2,
wherein each of the front leakage-preventing gather and the rear leakage-preventing gather includes a side sheet partially fixed to the front surface sheet or the back surface sheet, and a first elastic member disposed along an inner edge of the side sheet in the lateral direction and joined to the side sheet in a state of being stretched in the longitudinal direction, and
the first elastic member of the rear leakage-preventing gather is colored in a color different from the front surface sheet so as to be visibly recognizable through the side sheet.

9. The absorbent article for a pet according to claim 8, wherein an effective length of the first elastic member is ¼ or more of an entire length of the body portion in the longitudinal direction.

10. The absorbent article for a pet according to claim 8, wherein an inner edge of the side sheet of the rear leakage-preventing gather in the lateral direction is not joined to the front surface sheet over the entire length in the longitudinal direction.

11. The absorbent article for a pet according to claim 8, wherein an inner edge of the side sheet of the rear leakage-preventing gather in the lateral direction is joined to the front surface sheet in a state of being folded back outward in the lateral direction at both end portions in the longitudinal direction.

12. The absorbent article for a pet according to claim 3,
wherein the side sheet includes a fixed portion joined to the front surface sheet or the back surface sheet, and a rising portion disposed inside the fixed portion in the lateral direction and capable of rising with an inner edge of the fixed portion as a starting point, and
a height of the raised rising portion is larger than a width between the inner edge of the fixed portion and the rear edge in the lateral direction.

13. The absorbent article for a pet according to claim 12,
wherein the back surface sheet includes a liquid-impermeable back surface film and a back surface nonwoven fabric disposed on the non-skin surface side with respect to the back surface film, and a width of the back surface film is narrower than a width of the back surface nonwoven fabric in the lateral direction, and
a height of the raised rising portion is larger than a width between the inner edge of the fixed portion and an outer edge of the back surface film in the lateral direction.

14. The absorbent article for a pet according to claim 8, further comprising:
a pair of second elastic members disposed outside the first elastic member in the lateral direction and extending along the front edge and the rear edge of the body portion, respectively,
wherein the second elastic member extending along the rear edge, of the pair of second elastic members, is colored in a color different from the front surface sheet.

15. The absorbent article for a pet according to claim 14,
wherein the back surface sheet includes a liquid-impermeable back surface film and a back surface nonwoven fabric disposed on the non-skin surface side with respect to the back surface film, and a width of the back surface film is narrower than a width of the back surface nonwoven fabric in the lateral direction, and
the pair of second elastic members is joined to the back surface nonwoven fabric outside the back surface film in the lateral direction so as to be visibly recognizable from the non-skin surface side through the back surface nonwoven fabric.

16. The absorbent article for a pet according to claim 2, wherein at least a part of the rear leakage-preventing gather is housed in a package in a folded state so as to be visibly recognizable from an outside.
